(19) United States
(12) Patent Application Publication
(10) Pub. No.: US 2022/0088343 A1
(43) Pub. Date: Mar. 24, 2022

(54) METHOD FOR INDUCING SLEEP WITH SOUNDS AND SOUND PLAYBACK DEVICE PERFORMING THE SAME

(71) Applicant: PixArt Imaging Inc., Hsin-Chu County (TW)

(72) Inventors: Kuo-Wei KAO, Taipei (TW); Jing-Wei LI, Taipei (TW); Yu-Chieh HUANG, Taipei (TW); Kuo-Ping YANG, Taipei (TW)

(21) Appl. No.: 17/235,980

(22) Filed: Apr. 21, 2021

(30) Foreign Application Priority Data
Sep. 24, 2020 (CN) 202011016865.9

(51) Int. Cl. A61M 21/00

(52) U.S. Cl. CPC A61M 21/00; A61M 2021/0083; A61M 2205/18; A61M 2021/0027

(57) ABSTRACT

A method for inducing sleep with sounds includes the steps of: calculating and obtaining N sleep cycles based on an estimated sleep time of a user, where 3≤N≤15, and playing inducing sounds within at least three sleep cycles, wherein the inducing sounds include a first stage, a second stage, a third stage and a fourth stage. The method can be used for improving the sleep quality of a user.

### BACKGROUND OF THE INVENTION

### 1. Field of the Invention

The present invention relates to a method for inducing sleep with sounds and a sound playback device for performing the method, and more particularly, to a method and a sound playback device for inducing sleep in a user by means of playing binaural beats so as to improve the sleep quality of the user.

### 2. Description of the Related Art

Adequate sleep is critical for the mental and physical health and development of humans. However, along with progress in technology and civilization, the factors affecting sleep in humans continue to multiply such that sleep disorders and insomnia have become disturbing conditions of modern people, and particularly in the elderly, sleep disorders and insomnia can be severe.

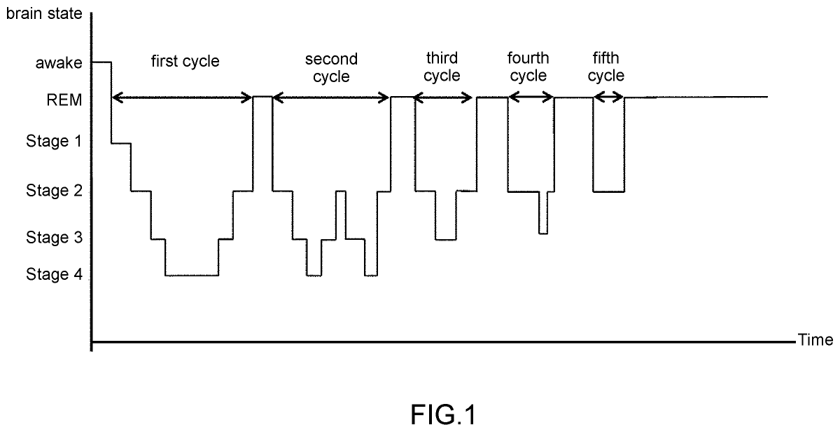

According to scientific discoveries, human brain waves exhibit repeated cyclic changes during sleep. FIG. 1 shows a schematic diagram of the brainwave states of a human in a sleep state. As shown in FIG. 1, during sleep, the human brain undergoes five stages in the sequence of a rapid eye movement (REM) stage, stage 1, stage 2, stage 3 and stage 4, in each of which the brainwaves are slightly different. These five stages form one sleep cycle and cyclically repeat during sleep, forming a process of a plurality of repetitively occurring sleep cycles.

During stage 3 and stage 4, as shown in FIG. 1, humans are in deep sleep. Researchers point out that body tissue repair, immune system strengthening, and memory consolidation occur in humans during this deep sleep period, and thus the deep sleep period is an essential key that affects the sleep quality of humans. Current research indicates that playing binaural beats with a frequency difference equivalent to a frequency of the delta brainwave for a user to listen to in stage 2 facilitates prolongation of the duration of deep sleep.

U.S. Pat. No. 9,764,110 B2 discloses a binaural sleep-inducing system which is capable of generating binaural beats having a corresponding frequency difference according to brainwave states of different sleep stages of a user. However, the frequency difference of the binaural beats is abruptly adjusted only upon the detection and acquisition of the result of a brainwave state of the user, and it cannot be adjusted in a moderate incremental or decremental manner Because a user who uses the method above to help sleep usually has poor sleep quality, the user sometimes may feel that a next inducing sound is not at all heard so that sleep quality is undesirably affected, which needs to be improved.

### SUMMARY OF THE INVENTION

It is a major objective of the present invention to provide a method for inducing sleep by playing binaural beats so as to improve sleep quality.

It is another major objective of the present invention to provide a sound playback device for performing the above method.

To achieve the abovementioned objects, the method for inducing sleep with sounds is applied to a sound playback device, wherein the sound playback device can play an inducing sound having a frequency difference to a user. The method for inducing sleep with sounds includes the steps of: calculating and obtaining N sleep cycles based on an estimated sleep time of a user, where 3≤N≤15, and playing the inducing sound within at least three sleep cycles, wherein the inducing sound includes a first stage, a second stage, a third stage and a fourth stage. In the first stage, the frequency difference of the inducing sound decreases in a continuous moderate decremental manner from a first frequency difference to a second frequency difference, wherein the continuous moderate decremental manner is to decrease the frequency difference at an interval of no more than X seconds, where 0<X≤10, or to reduce the frequency difference by no more than Y hertz each time, where 0<Y≤1. In the second stage, the frequency difference of the inducing sound is kept at the second frequency difference. In the third stage, the frequency difference of the inducing sound increases in a continuous moderate incremental manner from the second frequency difference to a third frequency difference, wherein the continuous moderate incremental manner is to increase the frequency difference at an interval of no more than Z seconds, where 0<Z≤10, or to increase the frequency difference by no more than S hertz each time, where 0<S≤1. In the fourth stage, the frequency difference of the inducing sound is kept at the third frequency difference.

The sound playback device of the present invention can play an inducing sound having a frequency difference to a user. The sound playback device includes a speaker and a sound playback controller. The sound playback controller is electrically connected to the speaker and calculates and obtains N sleep cycles based on an estimated sleep time of the user, where 3≤N≤15, and controls the speaker to play the inducing sound within at least three sleep cycles. The inducing sound includes a first stage, a second stage, a third stage and a fourth stage. In the first stage, the frequency difference of the inducing sound decreases in a continuous moderate decremental manner from a first frequency difference to a second frequency difference, wherein the continuous moderate decremental manner is to decrease the frequency difference at an interval of no more than X seconds, where 0<X≤10, or is to reduce the frequency difference by no more than Y hertz each time, where 0<Y≤1. In the second stage, the frequency difference of the inducing sound is kept at the second frequency difference. In the third stage, the frequency difference of the inducing sound increases in a continuous moderate incremental manner from the second frequency difference to a third frequency difference, wherein the continuous moderate incremental manner is to increase the frequency difference at an interval of no more than Z seconds, where 0<Z≤10, or is to increase the frequency difference by no more than S hertz each time, where 0<S≤1. In the fourth stage, the frequency difference of the inducing sound is kept at the third frequency difference.

### DETAILED DESCRIPTION OF THE PREFERRED EMBODIMENT

These and other objects and advantages of the present invention will become apparent from the following descriptions of the accompanying drawings, which disclose several embodiments of the present invention. It is to be understood that the drawings are to be used for purposes of illustration only and not as a definition of the invention. In the drawings, wherein similar reference numerals denote similar elements throughout the several views:

FIG. 1 illustrates a schematic diagram of brainwave states of a human in a sleep state;

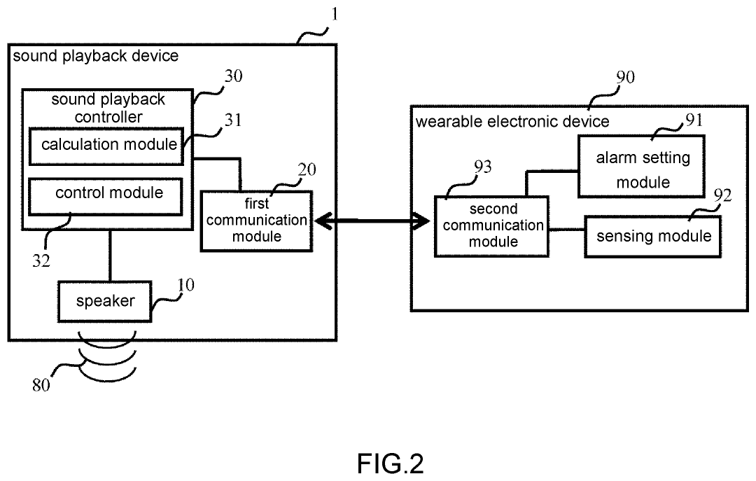

FIG. 2 illustrates a device structural drawing of a sound playback device according to an embodiment of the present invention;

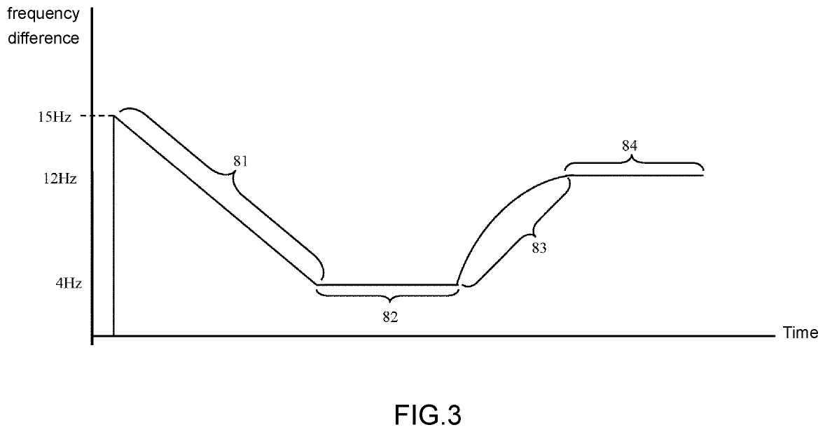

FIG. 3 illustrates a state of change in a frequency difference of inducing sounds according to an embodiment of the present invention; and

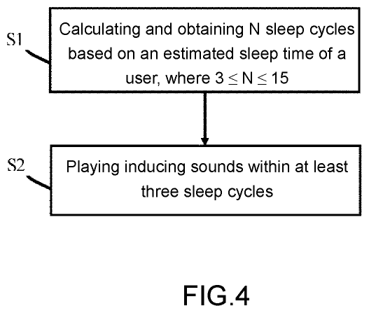

FIG. 4 illustrates a flowchart of a method for inducing sleep with sounds according to an embodiment of the present invention.

### DETAILED DESCRIPTION OF THE PREFERRED EMBODIMENT

Preferred embodiments are given below for the examiner to better understand the technical contents of the present invention.

Refer to FIG. 1 and FIG. 3. FIG. 1 illustrates a schematic diagram of brainwave states of a human in a sleep state. FIG. 2 illustrates a device structural drawing of a sound playback device according to an embodiment of the present invention. FIG. 3 illustrates a state of change in a frequency difference of inducing sounds according to an embodiment of the present invention.

As shown in FIG. 2, in an embodiment of the present invention, a sound playback device 1 of the present invention is capable of playing inducing sounds 80 having a frequency difference to a user for the user to listen to while asleep and thereby to improve the sleep quality of the user. In a specific embodiment, the inducing sounds 80 having a frequency difference are binaural beats; however, the present invention is not limited thereto, and the inducing sounds 80 may also be monaural beats.

In an embodiment of the present invention, the sound playback device 1 can be connected to and communicate with a wearable electronic device 90 in a wireless manner The wearable electronic device 90 includes an alarm setting module 91, a sensing module 92 and a second communication module 93. The alarm setting module 91 is configured for a user to set alarm reminder time data. When a reminder time recorded by the alarm reminder time data arrives, the wearable electronic device 90 sounds the alarm to remind the user. The sensing module 92 is configured to sense and obtain motion state data of the user, such as by sensing a body tossing state of the user. The second communication module 93 is electrically connected to the alarm setting module 91 and the sensing module 92 and is configured to send the alarm reminder time data and the motion state data to the sound playback device 1. It should be noted that the sound playback device 1 herein is not necessarily connected to and communicating with the wearable electronic device 90; in other embodiments, the wearable electronic device 90 can also be replaced by another electronic device having alarm setting and body motion state sensing functions, e.g., a smartphone. Furthermore, the alarm setting module 91 and the sensing module 92 can also be directly configured in the sound playback device 1.

As shown in FIG. 2, in an embodiment of the present invention, the sound playback device 1 of the present invention includes a speaker 10, a first communication module 20 and a sound playback controller 30.

In an embodiment of the present invention, the first communication module 20 is connected to and communicates with the second communication module 93 and is configured to receive the alarm reminder time data and the motion state data sent from the second communication module 93.

In an embodiment of the present invention, the sound playback controller 30 is electrically connected to the speaker 10 and the first communication module 20 and includes a calculation module 31 and a control module 32.

It should be noted that the above calculation module 31 and control module 32 can be accomplished by hardware devices, software programs, firmware or combinations thereof, and that they can also be configured in the form of circuit loops or other suitable formats; further, each of the modules can be configured either in an independent form or in a combined form. In one preferred embodiment, each of the modules is a software program stored in a storage unit (not shown in figures) of the sound playback controller 30, and a processing unit (not shown in figures) of the sound playback controller 30 will execute each module to achieve the purpose of the present invention. Moreover, the embodiment disclosed herein only describes a preferred embodiment of the present invention. To avoid redundant description, not all possible variations and combinations are described in detail in this specification. However, those skilled in the art will understand that the above modules or components are not all necessary parts, and that in order to implement the present invention, other more detailed known modules or components might also be included. It is possible that each module or component can be omitted or modified depending on different requirements, and it is also possible that other modules or components might be disposed between any two modules.

The calculation module 31 of the sound playback controller 30 is configured to calculate and obtain N sleep cycles based on an estimated sleep time of the user, where 3≤N≤15. The control module 32 of the sound playback controller 30 is configured to control the speaker 10 to play the inducing sounds 80 having a frequency difference within at least three sleep cycles.

In this embodiment, the estimated sleep time of the user is calculated and obtained by the calculation module 31 according to a predicted sleep time of the user and a predicted wake-up time of the user. The calculation module 31 determines the predicted sleep time of the user according to a body tossing state of the user. More specifically, the sensing module 92 of the wearable electronic device 90 can sense and obtain the motion state data of the user and send the motion state data to the sound playback device 1. The calculation module 31 of the sound playback controller 30 can determine the body tossing state of the user according to the motion state data. Once the calculation module 31 determines that the body of the user is in a non-wavering state, for example, the frequency of wavering is less than a predetermined value or the amplitude of wavering is less than a predetermined value, it is determined that the user has entered a sleep state, and this time is used as the predicted sleep time of the user. In a specific embodiment, the sensing module 92 senses and obtains the motion state data of the user once per minute; however, the present invention is not limited thereto.

It should be noted that the module for determining the body tossing state of the user is not limited to the calculation module 31. In other embodiments, the sensing module 92 can also be used to determine the body tossing state of the user and to send a notification signal to the sound playback device 1 upon determining that the body of the user is in a non-wavering state for the calculation module 31 to use the time of receiving the notification signal as the predicted sleep time of the user.

In this embodiment, the predicted wake-up time of the user is determined according to an alarm reminder time set by the user. More specifically, the alarm setting module 91 of the wearable electronic device 90 sends the alarm reminder time data obtained from the user setting to the sound playback device 1. Once the calculation module 31 of the sound playback controller 30 receives the alarm reminder time data, the reminder time recorded by the alarm reminder time data is set as the predicted wake-up time of the user.

The calculation module 31 of the sound playback controller 30, after sequentially obtaining information of the predicted sleep time and the alarm reminder time, calculates and obtains an estimated sleep time of the user according to the two and calculates and obtains N sleep cycles based on the predicted sleep time. The value of N varies according to the duration of the sleep time. In general, a normal sleep cycle lasts for about 90 minutes, and for a normal sleep time of 7 hours, N is approximately 4 to 5. The control module 32 of the sound playback controller 30, after obtaining information of the estimated sleep time and the N sleep cycles, controls the speaker 10 to play the inducing sounds 80 having a frequency difference within at least three sleep cycles.

As shown in FIG. 3, the inducing sounds 80 played by the speaker 10 under the control of the control module 32 include a first stage 81, a second stage 82, a third stage 83 and a fourth stage 84.

In the first stage 81, the frequency difference of the inducing sounds 80 decreases in a continuous moderate decremental manner from a first frequency difference to a second frequency difference. The so-called “continuous moderate decremental manner” is to decrease the frequency difference at an interval of no more than X seconds, where 0<X≤10, or is to reduce the frequency difference by no more than Y hertz each time, where 0<Y≤1. In a specific embodiment, the frequency difference of the inducing sounds 80 in the first stage 81 decreases by 0.006 hertz at an interval of every 1 second; that is, the frequency difference is adjusted moderately in a proportional linear manner during the process of decreasing. However, the present invention is not limited to decreasing in such proportional linear manner Due to the adjustment of the frequency difference of the inducing sounds 80 in a continuous moderate decremental manner, the user does not hear abrupt changes, so the user experience is improved.

As shown in FIG. 1, when the human brain state is in stage 1 and stage 2, the brain produces alpha brainwaves having a frequency of approximately 8 to 13 hertz. Thus, in an embodiment of the present invention, the first frequency difference is a hertz, where 10≤a≤20; in a specific embodiment, a is 15; however, the present invention is not limited thereto. Moreover, when the human brain state is in stage 3 and stage 4, the brain produces delta brainwaves having a frequency of approximately 4 to 8 hertz. Thus, in an embodiment of the present invention, the second frequency difference is b hertz, where 2≤b≤4; in a specific embodiment, b is 4; however, the present invention is not limited thereto.

In the second stage 82, the frequency difference of the inducing sounds 80 is kept at the second frequency difference.

In the third stage 83, the frequency difference of the inducing sounds 80 increases in a continuous moderate incremental manner from the second frequency difference to a third frequency difference. The so-called “continuous moderate incremental” manner is to increase the frequency differences at an interval of no more than Z seconds, where 0<Z≤10, or to increase the frequency difference by no more than S hertz each time, where 0<S≤1. As shown in FIG. 3, in a specific embodiment, the incremental amplitude of the frequency difference gradually changes from large to small during the incremental process; that is, the frequency difference is not adjusted in a proportional linear manner in the incremental process. However, the continuous moderate incremental manner of the present invention is not limited thereto. Similarly, by adjusting the frequency difference of the inducing sounds 80 in a continuous moderate incremental manner, the user does not hear abrupt changes, so the user experience is improved.

As shown in FIG. 1, in the later stage of a sleep period, the human brain state is in stage 1 and stage 2, and the brain produces alpha brainwaves. Thus, in an embodiment of the present invention, the third frequency difference is c hertz, where 10≤c≤20; in a specific embodiment, c is 12; however, the present invention is not limited thereto.

In the fourth stage 84, the frequency difference of the inducing sounds 80 is kept at the third frequency difference.

Next, referring to FIG. 1 to FIG. 4, FIG. 4 shows a flowchart of steps of a method for inducing sleep with sounds according to an embodiment of the present invention. The steps in FIG. 4 are described with reference to FIG. 1 to FIG. 3 below. It should be noted that, although the sound playback device 1 is used above as an example to describe the method for inducing sleep with sounds of the present invention, the method for inducing sleep with sounds of the present invention is not limited to being applied to a device having the same structure as the sound playback device 1 above.

As shown in FIG. 4, first of all, performing step S1: calculating and obtaining N sleep cycles based on an estimated sleep time of a user, where 3≤N≤15.

In an embodiment of the present invention, the sound playback controller 30 of the sound playback device 1 can calculate and obtain N sleep cycles based on an estimated sleep time of a user, where 3≤N≤15. The value of N varies according to the duration of the sleep time. The estimated sleep time of the user is calculated and obtained according to a predicted sleep time of the user and a predicted wake-up time of the user.

The predicted sleep time is determined according to a body tossing state of the user. In a specific embodiment, the sound playback controller 30 can determine the body tossing state of the user according to the motion state data sent from a wearable electronic device 90. When the sound playback controller 30 determines that the body of the user is a non-wavering state, for example, the wavering frequency is less than a predetermined value or the wavering amplitude is less than a predetermined value, it is defined that the user at this point in time has entered a sleep state, and the current time is used as the predicted sleep time of the user.

The predicted wake-up time is determined according to an alarm reminder time set by the user. In a specific embodiment, the sound playback controller 30 can receive the alarm reminder time data from the wearable electronic device 90 and set the reminder time recorded by the alarm reminder time data as the predicted wake-up time, wherein the alarm reminder time data is generated by the user operating the wearable electronic device 90.

Performing step S2: playing inducing sounds within at least three sleep cycles.

Once information of the estimated sleep time and the N sleep cycles is obtained, the sound playback controller 30 controls the speaker 10 to play the inducing sounds 80 having a frequency difference within at least three sleep cycles. As shown in FIG. 3, the inducing sounds 80 played by the speaker 10 under the control of the control module 32 include a first stage 81, a second stage 82, a third stage 83 and a fourth stage 84.

In the first stage 81, the frequency difference of the inducing sounds 80 decreases in a continuous moderate decremental manner from a first frequency difference to a second frequency difference. The so-called “continuous moderate decremental” manner is to reduce the frequency difference at an interval of no more than X seconds, where 0<X≤10, or is to reduce the frequency difference by no more than Y hertz each time, where 0<Y≤1. In a specific embodiment, the frequency difference of the inducing sounds 80 in the first stage 81 decreases by 0.006 hertz at an interval of every 1 second; that is, the frequency difference is adjusted moderately in a proportional linear manner during the decreasing process. However, the present invention is not limited to decreasing in such proportional linear manner In an embodiment of the present invention, the first frequency difference is a hertz, where 10≤a≤20; the second frequency difference is b hertz, where 2≤b≤4; however, the present invention is not limited thereto.

In the second stage 82, the frequency difference of the inducing sounds 80 is kept at the second frequency difference.

In the third stage 83, the frequency difference of the inducing sounds 80 increases in a continuous moderate incremental manner from the second frequency difference to a third frequency difference. The so-called “continuous moderate incremental” manner is to increase the frequency differences at an interval of no more than Z seconds, where 0<Z≤10, or to increase the frequency difference by no more than S hertz each time, where 0<S≤1. As shown in FIG. 3, in a specific embodiment, the incremental amplitude of the frequency difference gradually changes from large to small during the incremental process; that is, the frequency difference is not adjusted by a proportional linear manner in the incremental process. However, the continuous moderate incremental manner of the present invention is not limited thereto. In an embodiment of the present invention, the third frequency difference is c hertz, where 10≤c≤20.

In the fourth stage 84, the frequency difference of the inducing sounds 80 is kept at the third frequency difference.

It is to be understood from the foregoing description of the embodiments that the method for inducing sleep with sounds and the sound playback device 1 of the present invention can play the inducing sounds 80 based on brainwaves produced by the brain in different states, thereby facilitating prolongation of the duration of deep sleep to improve the sleep quality of users. Moreover, the frequency difference of the inducing sounds 80 of the present invention is adjusted in “continuous moderate decremental” and “continuous moderate incremental” manners such that the user does not hear abrupt changes during the adjustment process, so the user experience is improved.

While the present invention has been described by way of example and in terms of the embodiments above, it is to be understood that the present invention is not limited thereto. Slight changes and modifications can be made by a person skilled in the art without departing from the spirit and scope of the present invention, and such changes and modifications shall fall within the scope of the patent right claimed by the present invention. Therefore, the legal protection for the present invention shall be defined by the appended claims

What is claimed is:

1. A method for inducing sleep with sounds, applied to a sound playback device, the sound playback device configured to play an inducing sound having a frequency difference to a user, the method comprising the steps of:
calculating and obtaining N sleep cycles based on an estimated sleep time of the user, where 3≤N≤15; and
playing the inducing sound within at least three sleep cycles, the inducing sound comprising a first stage, a second stage, a third stage and a fourth stage;
wherein, in the first stage, the frequency difference of the inducing sound decreases in a continuous moderate decremental manner from a first frequency difference to a second frequency difference, wherein the continuous moderate decremental manner is to decrease the frequency difference at an interval of no more than X seconds, where 0<X≤10, or to reduce the frequency difference by no more than Y hertz each time, where 0<Y≤1;
in the second stage, the frequency difference of the inducing sound is kept at the second frequency difference;
in the third stage, the frequency difference of the inducing sound increases in a continuous moderate incremental manner from the second frequency difference to a third frequency difference, wherein the continuous moderate incremental manner is to increase the frequency difference at an interval of no more than Z seconds, where 0<Z≤10, or to increase the frequency difference by no more than S hertz each time, where 0<S≤1; and
in the fourth stage, the frequency difference of the inducing sound is kept at the third frequency difference.

2. The method as claimed in claim 1, wherein the first frequency difference is a hertz, the second frequency difference is b hertz, and the third frequency difference is c hertz, where 10≤a≤20, 2≤b≤4, and 10≤c≤20.

3. The method as claimed in claim 1, wherein the estimated sleep time is calculated and obtained according to a predicted sleep time and a predicted wake-up time.

4. The method as claimed in claim 3, wherein the predicted wake-up time is determined according to an alarm reminder time set by the user.

5. The method as claimed in claim 3, wherein the predicted sleep time is determined according to a body tossing state of the user, and the predicted wake-up time is determined by an alarm reminder time set by the user.

6. A sound playback device, configured to play an inducing sound having a frequency difference to a user, the sound playback device comprising:
a speaker; and
a sound playback controller, electrically connected to the speaker, the sound playback controller calculating and obtaining N sleep cycles based on an estimated sleep time of the user, where 3≤N≤15, and controlling the speaker to play the inducing sound within at least three sleep cycles, the inducing sound comprising a first stage, a second stage, a third stage and a fourth stage;
wherein, in the first stage, the frequency difference of the inducing sound decreases in a continuous moderate decremental manner from a first frequency difference to a second frequency difference, wherein the continuous moderate decremental manner is to decrease the frequency difference at an interval of no more than X seconds, where 0<X≤10, or to reduce the frequency difference by no more than Y hertz each time, where 0<Y≤1;
in the second stage, the frequency difference of the inducing sound is kept at the second frequency difference;
in the third stage, the frequency difference of the inducing sound increases in a continuous moderate incremental manner from the second frequency difference to a third frequency difference, wherein the continuous moderate incremental manner is to increase the frequency difference at an interval of no more than Z seconds, where 0<Z≤10, or to increase the frequency difference by no more than S hertz each time, where 0<S≤1; and
in the fourth stage, the frequency difference of the inducing sound is kept at the third frequency difference.

7. The sound playback device as claimed in claim 6, wherein the first frequency difference is a hertz, the second frequency difference is b hertz, and the third frequency difference is c hertz, where 10≤a≤20, 2≤b≤4, and 10≤c≤20.

8. The sound playback device according to claim 6, wherein the estimated sleep time is calculated and obtained according to a predicted sleep time and a predicted wake-up time.

9. The sound playback device as claimed in claim 8, wherein the predicted wake-up time is determined according to an alarm reminder time set by the user.

10. The sound playback device as claimed in claim 8, wherein the predicted sleep time is determined according to a body tossing state of the user, and the predicted wake-up time is determined by an alarm reminder time set by the user.
